(19) United States
(12) Patent Application Publication
(10) Pub. No.: US 2005/0139517 A1
(43) Pub. Date: Jun. 30, 2005

(54) Reactor having reduced pressure drop and use thereof

(76) Inventors: Thomas J. Waddick, League City, TX (US); Donald James Norris, Houston, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 10/746,001

(22) Filed: Dec. 24, 2003

(51) Int. Cl. C10G 11/10; C10G 73/38; C10G 45/00; C07C 2/66; C07C 5/22; C07C 1/20; B01J 8/04; B01J 10/00

(52) U.S. Cl. 208/113; 208/108; 208/27; 585/477; 585/470; 585/446; 585/407; 208/213; 585/639; 422/188; 422/190; 422/195

(57) ABSTRACT

A vertical reactor having reduced pressure drop across the catalytic bed of the reactor. The reactor finds particular application in the treatment of feeds such as the conversion of organic compounds and the removal of undesired components, e.g., sulfur, from organic feeds.

### FIELD OF THE INVENTION

This invention relates to a reactor having reduced pressure drop across the catalytic bed of the reactor. This invention also relates to the use of the reactor in the treatment of feeds such as the conversion of organic compounds and the removal of undesired components from feeds.

### BACKGROUND OF THE INVENTION

The chemical and petroleum industry commonly uses single phase reactors to process fluids across a fixed catalytic bed for the hydroprocessing of feed streams. Many times the effectiveness of these reactors is limited by the amount of pressure drop across the catalytic bed because high pressure drop across the catalytic bed of the reactor reduces the throughput capacity of the reactor. Also, high pressure drop across the catalytic bed of the reactor can cause the catalyst particles to crush which results in the restriction in the flow of fluid through the reactor leading to even higher pressure drops.

The pressure drop of the reactor is often the limiting factor in throughput capacity. This can result in significant costs for expansion or de-bottlenecking of existing facilities and limit the use of existing equipment in new or retrofitted installations. Traditional techniques for controlling pressure drop include adding compressions or pumping equipment to equipment and installation of larger diameter reactors and/or piping. Another technique for controlling pressure drop, as disclosed in U.S. Pat. No. 5,837,128, involves grading the catalyst particles by pressure drop and then loading the particles into the reactor with the particles having the lowest pressure drop near the inlet and particles having the highest pressure drop near the outlet.

### SUMMARY OF THE INVENTION

In accordance with the present invention, there is provided a vertical reactor having reduced pressure drop across its catalytic bed. The reactor comprises:
(a) a vertical elongated vessel having an upper portion, a lower portion and a middle portion,
(b) said upper portion containing a first catalyst region and having a first conduit proximal to the top of said upper portion,
(c) said lower portion containing a second catalyst region and having a second conduit proximal to the bottom of said lower portion,
(d) said middle portion having a distributor fluidly connected to said upper portion, to said lower portion, and to a third conduit proximal to the mid-point of said middle portion and, optionally containing a filler region separating said first catalyst region and said second catalyst region,
(e) wherein said vertical reactor is capable of either:
   (i) accepting a feedstock comprising said organic compounds through said first conduit and said second conduit, and removing effluent from said vessel through said third conduit, or
   (ii) accepting a feedstock comprising said organic compounds through said third conduit, and removing effluent from said vessel through said first conduit and said second conduit.

In one embodiment of the present invention, the vertical reactor is configured to accept feedstock through the upper and lower portions of the vessel and remove effluent through the middle portion of the vessel. In this embodiment, the reactor comprises:
(a) a vertical elongated vessel having an upper portion, a lower portion and a middle portion,
(b) said upper portion containing a first catalyst region and having a first inlet proximal to the top of said upper portion,
(c) said lower portion containing a second catalyst region and having a second inlet proximal to the bottom of said lower portion, and,
(d) said middle portion having a distributor fluidly connected to said upper portion, to said lower portion, and to an outlet proximal to the mid-point of said middle portion and, optionally containing a filler region separating said first catalyst region and said second catalyst region.

In another embodiment of the present invention, the vertical reactor is configured to accept feedstock through the middle portion of the vessel and remove effluent through the upper and lower portions of the vessel. In this embodiment, the reactor comprises:
(a) a vertical elongated vessel having an upper portion, a lower portion and a middle portion,
(b) said upper portion containing a first catalyst region and having a first outlet proximal to the top of said upper portion,
(c) said lower portion containing a second catalyst region and having a second outlet proximal to the bottom of said lower portion, and,
(d) said middle portion having a distributor fluidly connected to said upper portion, to said lower portion, and to an inlet proximal to the mid-point of said middle portion and, optionally containing a filler region separating said first catalyst region and said second catalyst region.

The vertical reactor of the present invention finds particular application in the treatment of feeds such as the conversion of organic compounds and the removal of undesired components from feeds, e.g., the desulfurization of hydrocarbon streams (desulfurization of streams containing containing benzene, toluene, or mixtures thereof), and the adsorption of molecular species.

### BRIEF DESCRIPTION OF THE Drawings

FIG. 1 illustrates a vertical reactor with a distributor having a circumferential wall design.

FIG. 2 is a sectional view of the distributor used in the vertical reactor illustrated in FIG. 1.

FIG. 3 illustrates a vertical reactor with a distributor positioned concentrically in the middle portion of the reactor vessel.

FIG. 4 is a sectional view of the distributor used in the vertical reactor illustrated in FIG. 3.

### DETAILED DESCRIPTION OF THE INVENTION

The benefit of the present invention with respect to single phase flow can be shown by reference to the Ergun equation, which is set forth below.

$$\frac{Δ P}{L} = K Re ( 150 + 1.75 Re ) {( \frac{1 - ɛ}{ɛ} )}^{3} ( \frac{{μ}^{2}}{ρ {D}_{p}^{3}} )$$

Where:

$$\frac{Δ P}{L} = \text{Pressure Drop,} psi \text{/} ft , of the bed$$

psi/ft, of the bed

$$Re = \frac{{WD}_{p}}{μ ( 1 - ɛ )} ,$$
dimensionless Reynolds Number
ε=Void fraction of bed, dimensionless
μ=Viscosity of fluid
K=Dimensionless constant based on units.
ρ=Fluid density
Dₚ=Equivalent particle diameter
W=Mass velocity of fluid (mass flow/cross section of reactor)

Reference to the Ergun equation shows that reducing the mass velocity of the fluid (W) by a factor of about 2, while holding all other factors constant, results in a pressure drop reduction by a factor of about 4. Also, reducing the length of bed through which the fluid flows by a factor of about 2 results in an overall pressure drop reduction of a factor of about 8, resulting in approximately a 87.5% pressure drop reduction across the catalyst in the reactor.

Retrofitting an existing vessel with one inlet and one outlet, where the flow through the existing vessel is restricted by the pressure drop across the packing material, can reduce the pressure drop to approximately 10 to 20% of its former amount. This will allow the bed to either handle additional fresh feed, up to about 2 or 3 times the original throughput, or operate longer before regeneration is required to reduce pressure drop back to an acceptable operating level.

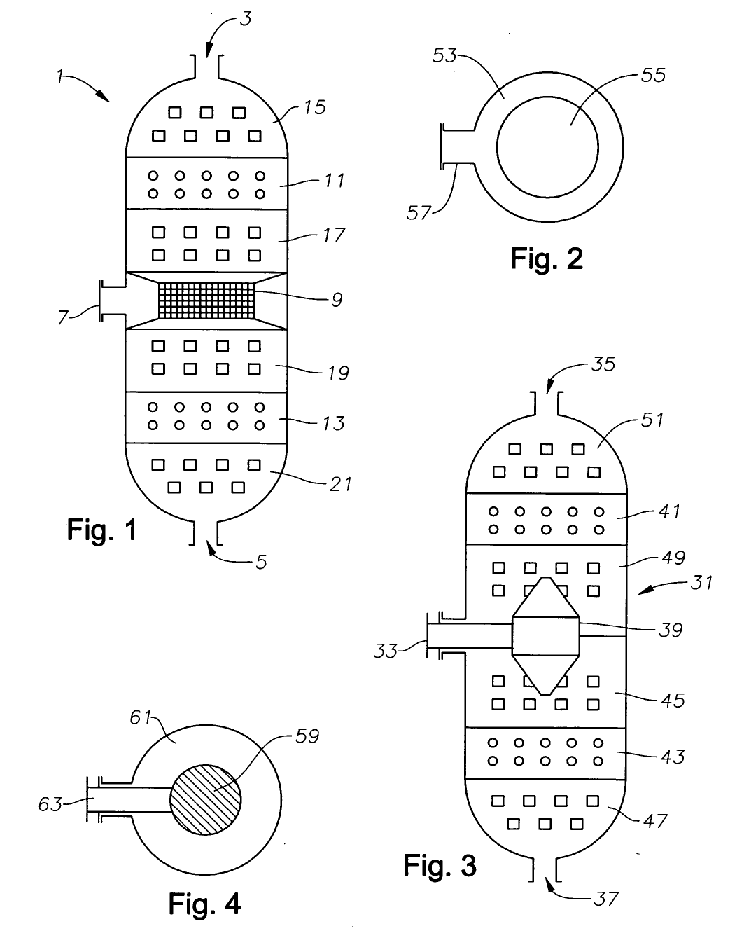

FIG. 1 illustrates a reactor according to the present invention. The reactor comprises vessel 1, inlet lines 3 and 5, outlet line 7, and distributor 9. Distributor 9 is an example of a distributor having a circumferential wall design. Vessel 1 is packed in catalyst regions 111 and 13 with appropriate catalyst to carry out the desired feed treatment. The catalyst in catalyst regions 11 and 13 can be the same or can be different. Usually catalyst is the same. Vessel 1 is also packed with inert filler in filler regions that separate the catalyst (filler regions 17 and 19) and upper filler region 15 and lower filler region 21. Although four filler regions are shown in FIG. 1, the vessel can contain more than four filler regions or less than four filler regions (including no filler regions). In the operation of the reactor, feed enters the vessel via lines 3 and 5 and is treated when passed through the catalyst in catalyst regions 111 and 13. The treated feed is then collected by distributor 9 which directs the treated feed to outlet line 7 where the feed exits the vessel. Although the operation of the reactor is described above as having feedstock being accepted through the upper and lower portions of the vessel (through inlet lines 3 and 5) and effluent being removed from the vessel through the middle portion of the vessel (through outlet line 7), the reactor can also be operated to have feedstock entering the reactor through the middle portion of the vessel and effluent leaving the reactor through the upper and lower portions of the vessel.

FIG. 3 illustrates a reactor according to the present invention. The reactor comprises vessel 31, inlet 33, outlet lines 35 and 37, and distributor 39. Distributor 39 is an example of a distributor having a concentric design. Vessel 31 is packed in catalyst regions 41 and 43 with appropriate catalyst to carry out the desired feed treatment. The catalysts in catalyst regions 41 and 43 can be the same or can be different. Usually the catalyst is the same. Vessel 31 is also packed with inert filler in filler regions that separate the catalyst (filler regions 45 and 49) and upper filler region 51 and lower filler region 47. Although four filler regions are shown in FIG. 3, the vessel can contain more than four filler regions or less the four filler regions (including no filler regions). In the operation of the reactor, fluid enters the vessel via line 33 is treated when passed through the catalyst in catalyst regions 41 and 43. The treated fluid is then collected by distributor 39 which directs the treated fluid to outlet line 35 and 37 where the treated fluid exits the vessel. Although the operation of the reactor is described above as having feedstock being accepted through the middle portion of the vessel (through inlet line 33) and effluent being removed from the vessel through the upper and lower portions of the vessel (through outlet lines 35 and 37), the reactor can also be operated to have feedstock entering the reactor through the upper and lower portions of the vessel and effluent leaving the reactor through the middle portion of the vessel.

As used herein, the term “distributor” refers to a collection mechanism for balancing the distribution of the feed into the reactor and effluent out of the reactor. These collection mechanisms are known to persons skilled in the art and are located proximal to the middle portion of the vessel. Operation of the reactor of the present invention is not limited to any particular distributor design. The distributor shown in FIG. 1 has a circumferential wall design and the distributor shown in FIG. 3 has a concentric design. Circumferential wall design distributors comprise a collection ring surrounding the wall of the vessel with a void space proximal to the center of the collection ring. FIG. 2, a sectional view of the circumferential wall design distributor of FIG. 1, illustrates collection ring 53, void space 55 and conduit 57. In general, the circumferential distributor separates the upper and lower portions of the vessel. The circumferential distributor typically contains a central conduit 55 to allow solid particles such as catalyst and inert filler to pass from the upper portion of the vessel, into the lower portion of the vessel. The circumference of the central conduit 55 is typically made of mesh or contains other small openings which allow fluid connectivity, but does not allow solid particles to pass into the annular space 53. The annular space 53 is fluidly connected to the central conduit 55 and to an entrance or exit from the vessel (as the case may be), such as conduit line 7 of FIG. 1. Accordingly, the circumferential distributor may either receive treated feed from conduits 3 and 5 of FIG. 1, and distribute the treated feed to exit conduit 7, or in the alternative, receive the feed from conduit 7 and split and distribute the feed into the upper and lower portions of the vessel where the feed is treated and then removed from the vessel through exit conduits 3 and 5 respectively.

FIG. 4, a sectional view of the concentric design distributor of FIG. 3, illustrates collector 59, void space 61 and conduit 63. The concentric design distributor comprises a collection device 59, located proximal to the center of the vessel, and is made of mesh or contains other small openings to allow entry of fluids into or out of the collection device, but does not allow the passage of solid particles. The concentric distributor is connected to an entrance or exit from the vessel (as the case may be), such as conduit 33 of FIG. 3. The annular space 61 surrounding the collection device 59 allows solid particles such as catalyst and inert filler to pass from the upper portion of the vessel into the lower portion of the vessel.

An advantage of these type of distributors, e.g., concentric design distributors and circumferential wall design distributors are their ease of unloading of the catalyst, e.g., middle portion of the reactor is open to allow catalyst material to flow to the bottom of the reactor for unloading.

Examples of suitable filler material that can be included in the filler region of the reactor include inert ceramic balls or pellets, fired clay balls or pellets, and alumina balls or pellets.

The present invention finds particular applicable in reactors with flow in a single direction, said direction being oriented perpendicular to a given cross-section of the reactor. The reactor can be operated either in the liquid phase or vapor phase. The present invention can be applied to a new reactor as well as retrofitting to an existing reactor.

The vertical reactor of the present invention finds particular application in the treatment of feeds such as the conversion of organic compounds and the removal of undesired components from organic feeds.

Processes that find particular application include, as non-limiting examples, the following:

(A) Cracking of hydrocarbons with reaction conditions including a temperature of from about 300° C. to about 700° C., a pressure of from about 0.1 atmosphere (bar) to about 30 atmospheres and weight hourly space velocity of from about 0.1 hr⁻¹ to about 20 hr⁻¹.

(B) Dehydrogenating hydrocarbon compounds with reaction conditions including a temperature of from about 300° C. to about 700° C., a pressure of from about 0.1 atmosphere (bar) to about 10 atmospheres and weight hourly space velocity of from about 0.1 hr⁻¹ to about 20 hr⁻¹.

(C) Converting paraffins to aromatics with reaction conditions including from about 300° C. to about 700° C., a pressure of from about 0.1 atmosphere (bar) to about 60 atmospheres and weight hourly space velocity of from about 0.5 hr⁻¹ to about 400 hr⁻¹ and a hydrogen/hydrocarbon mole ratio of from about 0 to about 20.

(D) Converting olefins to aromatics, e.g., benzene, toluene and xylene, with reaction conditions including a temperature from about 100° C. to about 700° C., a pressure of from about 0.1 atmosphere (bar) to about 60 atmospheres, weight hourly space velocity of from about 0.5 hr⁻¹ to about 400 hr⁻¹, and a hydrogen/hydrocarbon mole ratio of from about 0 to about 20.

(E) Converting alcohols, e.g., methanol, or ethers, e.g., dimethylether, or mixtures thereof to hydrocarbons, including olefins and/or aromatics with reaction conditions including a temperature from about 275° C. to about 600° C., a pressure of from about 0.5 atmosphere (bar) to about 50 atmospheres, weight hourly space velocity of from about 0.5 hr⁻¹ to about 100 hr⁻¹.

(F) Isomerization of dialkyl substituted benzenes, e.g., xylenes. Typical reaction conditions including a temperature from about 230° C. to about 510° C., a pressure of from about 1 atmosphere to about 50 atmospheres, a weight hourly space velocity of from about 0.1 hr⁻¹ to about 200 hr⁻¹ and a hydrogen/hydrocarbon mole ratio of from 0 (no added hydrogen) to about 100.

(G) Alkylating aromatic hydrocarbons, e.g., benzene and alkylbenzenes in the presence of an alkylating agent, e.g., olefins, formaldehyde, alkyl halides and alcohols, with reaction conditions including a temperature from about 250° C. to about 500° C., a pressure of from about atmospheric to about 200 atmospheres, weight hourly space velocity of from about 2 hr⁻¹ to about 2000 hr⁻¹ and an aromatic hydrocarbon/alkylating agent mole ratio of from about 1/1 to about 20/1.

(H) Transalkylating aromatic hydrocarbons in the presence of polyalkylaromatic hydrocarbons with reaction conditions including a temperature from about 340° C. to about 500° C., a pressure of from about atmospheric to about 200 atmospheres, weight hourly space velocity of from about 10 hr⁻¹ to about 1000 hr⁻¹, and an aromatic hydrocarbon/polyalkylaromatic hydrocarbon mole ratio of from about 1/1 to about 16/1.

(I) Dewaxing of hydrocarbons by selectively removing straight chain paraffins. The reaction conditions are dependent in large measure on the feed used and upon the desired pour point. Typical reaction conditions include a temperature between about 200° C. and 450° C., a pressure up to 3,000 psig and a liquid hourly space velocity from about 0.1 to about 20.

(J) Alkylation of a reformate containing substantial quantities of benzene and toluene with fuel gas containing short chain olefins (e.g., ethylene and propylene) to produce mono- and dialkylates. Preferred reaction conditions include temperatures from about 100° C. to about 250° C., a pressure of from about 100 to about 800 psig, a WHSV-olefin from about 0.4 hr⁻¹ to about 0.8 hr⁻¹, a WHSV-reformate of from about 1 hr⁻¹ to about 2 hr⁻¹ and, optionally, a gas recycle from about 1.5 to 2.5 vol/vol fuel gas feed.

(K) Alkylation of phenols with olefins or equivalent alcohols to provide long chain alkyl phenols. Typical reaction conditions include temperatures from about 100° C. to about 250° C., pressures from about 1 to 300 psig and total WHSV of from about 2 hr⁻¹ to about 10 hr⁻¹.

(L) Reaction of alcohols with olefins to produce mixed ethers, e.g., the reaction of methanol with isobutene and/or isopentene to provide methyl-t-butyl ether (MTBE) and/or t-amyl methyl ether (TAME). Typical conversion conditions include temperatures from about 20° C. to about 200° C., pressures from 2 to about 200 atm, WHSV (gram-olefin per hour gram-catalyst) from about 0.1 hr⁻¹ to about 200 hr⁻¹ and an alcohol to olefin molar feed ratio from about 0.1/1 to about 5/1.

(M) Disproportionation of alkyl aromatics, e.g., the disproportionation of toluene to make benzene and paraxylene and the disproportionation of cumene to make benzene and diisopropylbenzene. Typical reaction conditions include a temperature of from about 200° C. to about 760° C., a pressure of from about atmospheric to about 60 atmosphere (bar), and a WHSV of from about 0.1 hr⁻¹ to about 30 hr⁻¹.

(N) Selectively separating hydrocarbons by adsorption of the hydrocarbons. Examples of hydrocarbon separation include xylene isomer separation and separating olefins from a feed stream containing olefins and paraffins.

(O) Oligomerization of straight and branched chain olefins having from about 2 to about 5 carbon atoms. The oligomers which are the products of the process are medium to heavy olefins which are useful for both fuels, i.e., gasoline or a gasoline blending stock, and chemicals. The oligomerization process is generally carried out by contacting the olefin feedstock in a gaseous state phase with a catalyst at a temperature in the range of from about 250° C. to about 800° C., a LHSV of from about 0.2 to about 50 and a hydrocarbon partial pressure of from about 0.1 to about 50 atmospheres. Temperatures below about 250° C. may be used to oligomerize the feedstock when the feedstock is in the liquid phase when contacting the catalyst. Thus, when the olefin feedstock contacts the catalyst in the liquid phase, temperatures of from about 10° C. to about 250° C. may be used.

(P) Dealkylation of alkylaromatic compounds. In the case of ethylbenzene, the ethylbenzene can be converted to benzene and ethane. Typical reaction conditions including a temperature from about 230° C. to about 510° C., a pressure of from about 1 atmosphere to about 50 atmospheres, a weight hourly space velocity of from about 0.1 hr⁻¹ to about 200 hr⁻¹ and a hydrogen/hydrocarbon mole ratio of from 0 (no added hydrogen) to about 100.

(Q) Isomerization of ethylbenzene to form xylenes. Exemplary conditions include a temperature from about 300° C. to about 550° C., a pressure of from about 50 to 500 psig, and a LHSV of from about 1 to about 20.

(R) Isomerization of dialkylnaphthalene, e.g., dimethylnaphthalene, to form a mixture of isomers. Of the dimethylnapthalene isomers, 2,6-dimethylnapthalene is a key intermediate in the production of 2,6-napthalenedicarboxylic acid, a valuable monomer for specialty polyester manufacture. Typical reaction conditions including a temperature from about 230° C. to about 510° C., a pressure of from about 1 atmosphere to about 50 atmospheres, a weight hourly space velocity of from about 0.1 hr⁻¹ to about 200 hr⁻¹ and a hydrogen/hydrocarbon mole ratio of from 0 (no added hydrogen) to about 100.

(S) Disproportionation of mono-alkyl substituted naphthalenes, e.g., disproportionation of mono-methyl naphthalene to dimethyl-naphthalene and naphthalene. Typical reaction conditions including a temperature of from about 200° C. to about 760° C., a pressure of from about atmospheric to about 60 atmospheres and a weight hourly space velocity of from about 0.08 hr⁻¹ to about 20 hr⁻¹.

(T) Oxidation of alkyl substituted aromatic compounds, e.g., conversion of para-xylene to para-terephthalic acid and the conversion of cumene to phenol and acetone and the conversion of 2,6-dimethylnapthalene to 2,6-napthalenedicarboxylic acid.

(U) Desulfurization of an organic feed, e.g., desulfurization of a hydrocarbon stream, such as a stream containing benzene, toluene, or mixtures thereof.

(V) Denitrogenation of an organic feed such as the denitrogenation of a hydrocarbon feed comprising a petroluem fraction.

In general, the conversion conditions include a temperature from about 100° C. to about 760° C., a pressure of from about 0.1 atmosphere (bar) to about 200 atmospheres (bar), weight hourly space velocity of from about 0.08 hr⁻¹ to about 2000 hr⁻¹, and a hydrogen/organic, e.g., hydrocarbon compound, molar ratio of from about 0 to about 100.

The catalyst used in the reactor will depend on the process carried out in the reactor. Such catalysts will usually include amorphous metal oxides, such as alumina and silica, or crystalline molecular sieves.

Molecular sieves finding application include any of the naturally occurring or synthetic crystalline molecular sieves. Examples of these molecular sieves include large pore molecular sieves, intermediate pore size molecular sieves, and small pore molecular sieves. These materials are described in “Atlas of Zeolite Structure Types”, eds. Ch. Baerlocher, W. H. Meier, and D. H. Olson, Elsevier, Fifth Revised Edition, 2001, which is hereby incorporated by reference. A large pore molecular sieves generally has a pore size of at least about 7 Å and includes LTL, VFI, MAZ, MEI, FAU, EMT, OFF, *BEA, and MOR structure type molecular sieves (IUPAC Commission of Zeolite Nomenclature). Examples of large pore molecular sieves include mazzite, offretite, zeolite L, VPI-5, zeolite Y, zeolite X, omega, Beta, ZSM-3, ZSM-4, ZSM-18, ZSM-20, SAPO-37, and MCM-22. An intermediate pore size molecular sieves generally has a pore size from about 5 Å to about 7 Å and includes, for example, MFI, MEL, MTW, EUO, MTT, MFS, AEL, AFO, HEU, FER, and TON structure type zeolites (IUPAC Commission of Zeolite Nomenclature). Examples of intermediate pore size molecular sieves include ZSM-5, ZSM-11, ZSM-12, ZSM-22, ZSM-23, ZSM-34, ZSM-35, ZSM-385, ZSM-48, ZSM-50, ZSM-57, silicalite 1, and silicalite 2. A small pore size molecular sieves has a pore size from about 3 Å to about 5.0 Å and includes, for example, CHA, ERI, KFI, LEV, SOD, and LTA structure type zeolites (IUPAC Commission of Zeolite Nomenclature). Examples of small pore molecular sieves include ZK-4, ZSM-2, SAPO-34, SAPO-35, ZK-14, SAPO-42, ZK-21, ZK-22, ZK-5, ZK-20, zeolite A, hydroxysodalite, erionite, chabazite, zeolite T, gemlinite, ALPO-17, and clinoptilolite.

When the molecular sieve produced is a crystalline metallosilicate, the chemical formula of anhydrous crystalline metallosilicate can be expressed in terms of moles as represented by the formula: M₂/nO:W₂O₃:ZSiO₂, wherein M is selected from the group consisting of hydrogen, hydrogen precursors, monovalent, divalent, and trivalent cations and mixtures thereof; n is the valence of the cation and Z is a number of at least 2, preferably at least 3, said value being dependent upon the particular type of molecular sieve, and W is a metal in the anionic framework structure of the molecular sieve such as aluminum, gallium, boron, or iron.

When the molecular sieve produced has an intermediate pore size, the molecular sieve preferably comprises a composition having the following molar relationship:
X₂O₃:(n)YO₂,
wherein X is a trivalent element, such as aluminum, gallium, zinc, iron, and/or boron, Y is a tetravalent element such as silicon, tin, and/or germanium; and n has a value greater than 10, usually from about 20 to less than 20,000, more usually from 50 to 2,000, said value being dependent upon the particular type of molecular sieve and the trivalent element present in the molecular sieve.

When the molecular sieve is a gallosilicate intermediate pore size molecular sieve, the molecular sieve preferably comprises a composition having the following molar relationship:
Ga₂O₃:ySiO₂
wherein y is between about 20 and about 500, typically from 20 to 200. The molecular sieve framework may contain only gallium and silicon atoms or may also contain a combination of gallium, aluminum, and silicon.

The molecular sieve may be employed in combination with a binder material resistant to the temperature and other conditions employed in aromatic conversion processes. Such binder materials include synthetic or naturally occurring substances as well as inorganic materials such as clay, silica, alumina, and/or metal oxides. The latter may be either naturally occurring or in the form of gelatinous precipitates or gels including mixtures of silica and metal oxides. Naturally occurring clays include those of the montmorillonite and kaolin families, which families include the sub-bentonites and the kaolins commonly known as Dixie, McNamee-Georgia and Florida clays or others in which the main mineral constituent is halloysite, kaolinite, dickite, nacrite or anauxite. Such clays can be used in the raw state as originally mined or initially subjected to calcination, acid, treatment or chemical modification.

In addition to the foregoing materials, the molecular sieve may be composited with a porous matrix material, such as active carbon, carbon fiber, alumina, silica-alumina, silica-magnesia, silica-zirconia, silica-thoria, silica-beryllia, and silica-titania, as well as ternary compositions, such as silica-alumina-thoria, silica-alumina-zirconia, silica-alumina-magnesia and silica-magnesia-zirconia. Further, the molecular sieve may be composited with crystalline microporous molecular sieve material. Examples of such materials are disclosed in PCT Publication 96/16004, which is hereby incorporated by reference.

The relative proportions of molecular sieve and binder material will vary widely with the molecular sieve content ranging from between about 1 to about 99 percent by weight, more preferably in the range of about 10 to about 70 percent by weight of molecular sieve, and still more preferably from about 20 to about 50 percent.

The catalyst can include at least one hydrogenation/dehydrogenation metal. Examples of suitable hydrogenation/dehydrogenation metals include Group VIII metals (i.e., Pt, Pd, Ir, Rh, Os, Ru, Ni, Co and Fe), Group IVA metals (i.e., Sn and Pb), Group VA metals (i.e., Sb and Bi), and Group VIIB metals (i.e., Mn, Tc and Re). Noble metals (i.e., Pt, Pd, Ir, Rh, Os and Ru) are sometimes preferred. Reference to the metal or metals is intended to encompass such metal or metals in the elemental state (i.e. zero valent) or in some other catalytically active form such as an oxide, sulfide, halide, carboxylate and the like.

The reactor of the present invention finds particular application in sulfur removal and/or the saturation of olefins in a feed containing organic compounds, e.g., hydrocarbon feed containing benzene heartcut and hydrogen. A preferred catalyst for use in this process comprises an amorphous metal oxide support material, e.g., silica, alumina, or mixtures thereof and a hydrogenation/dehydrogenation metal such as nickel, molybdenum, or mixtures thereof.

Exemplary operating conditions for sulfur removal include a temperature of from about 200° C. to about 350° C., a pressure of from about atmospheric to about 60 atmospheres and a weight hourly space velocity of from about 0.08 hr⁻¹ to about 20 hr⁻¹.

1. A vertical reactor comprising:
(a) a vertical elongated vessel having an upper portion, a lower portion and a middle portion,
(b) said upper portion containing a first catalyst region and having a first conduit proximal to the top of said upper portion,
(c) said lower portion containing a second catalyst region and having a second conduit proximal to the bottom of said lower portion,
(d) said middle portion having a distributor fluidly connected to said upper portion, to said lower portion and to a third conduit proximal to the mid-point of said middle portion and, optionally containing a filler region separating said first catalyst region and said second catalyst region,
(e) wherein said vertical reactor is capable of either:
(i) accepting a feedstock comprising said organic compounds through said first conduit and said second conduit, and removing effluent from said vessel through said third conduit, or
(ii) accepting a feedstock comprising said organic compounds through said third conduit, and removing effluent from said vessel through said first conduit and said second conduit.

2. The reactor of claim 1, wherein said reactor further comprises a first catalyst located in said first catalyst region and a second catalyst located in said second catalyst region.

3. The reactor of claim 2, wherein said reactor includes said filler region and said filler region comprises inert material.

4. The reactor recited in claim 1, wherein the feedstock is accepted through said first conduit and said second conduit.

5. The reactor recited in claim 1, wherein the feedstock is accepted through said third conduit.

6. The reactor of claim 2, wherein said distributor has a circumferential wall design.

7. The reactor of claim 1, wherein said distributor has a concentric design.

8. The reactor of claim 2, wherein said first catalyst and said second catalyst is a crystalline molecular sieve or amorphous metal oxide.

9. The reactor of claim 8, wherein said first catalyst is the same as said second catalyst.

10. The reactor of claim 8, wherein said first catalyst is different from said second catalyst.

11. The reactor of claim 2, wherein said distributor is capable of allowing removal of said first catalyst and said second catalyst from the bottom of said lower portion of said vertical reactor.

12. A vertical reactor comprising:
(a) a vertical elongated vessel having an upper portion, a lower portion and a middle portion,
(b) said upper portion containing a first catalyst region and having a first inlet proximal to the top of said upper portion,
(c) said lower portion containing a second catalyst region and having a second inlet proximal to the bottom of said lower portion, and,
(d) said middle portion having a distributor fluidly connected to said upper portion, to said lower portion and to an outlet proximal to the mid-point of said middle portion and, optionally containing a filler region separating said first catalyst region and said second catalyst region.

13 The reactor of claim 12, wherein said reactor further comprises a first catalyst located in said first catalyst region and a second catalyst located in said second catalyst region.

14. A vertical reactor comprising:
(a) a vertical elongated vessel having an upper portion, a lower portion and a middle portion,
(b) said upper portion containing a first catalyst region and having a first outlet proximal to the top of said upper portion,
(c) said lower portion containing a second catalyst region and having a second outlet proximal to the bottom of said lower portion, and,
(d) said middle portion having a distributor fluidly connected to said upper portion, to said lower portion and to an inlet proximal to the mid-point of said middle portion and, optionally containing a filler region separating said first catalyst region and said second catalyst region.

15. The reactor of claim 14, wherein said reactor further comprises a first catalyst located in said first catalyst region and a second catalyst located in said second catalyst region.

16. A process for treating of a feed comprising organic compounds, said process comprising contacting said feed under sufficient conditions and in the presence of a catalyst contained in a vertical reactor, said vertical reactor comprising:
(a) a vertical elongated vessel having an upper portion, a lower portion and a middle portion,
(b) said upper portion containing a first catalyst and having a first conduit proximal to the top of said upper portion,
(c) said lower portion containing a second catalyst and having a second conduit proximal to the bottom of said lower portion,
(d) said middle portion having a distributor fluidly connected to said upper portion, to said lower portion and to a third conduit proximal to the mid-point of said middle portion and, optionally containing a filler region separating said first catalyst region and said second catalyst region,
(e) wherein said vertical reactor is capable of either:
(i) accepting a feedstock comprising said organic compounds through said first conduit and said second conduit, and removing effluent from said vessel through said third conduit, or
(ii) accepting a feedstock comprising said organic compounds through said third conduit, and removing effluent from said vessel through said first conduit and said second conduit.

17. The process of claim 16, wherein said reactor includes said filler region and said filler region comprises inert material.

18. The process recited in claim 17, wherein the feedstock is accepted through said first conduit and said second conduit.

19. The process recited in claim 16, wherein the feedstock is accepted through said third conduit.

20. The process of claim 16, wherein said distributor has a circumferential wall design.

21. The process of claim 16, wherein said distributor has a concentric design.

22. The process of claim 16, wherein said first catalyst and said second catalyst is a crystalline molecular sieve or amorphous metal oxide.

23. The process of claim 22, wherein said first catalyst is the same as said second catalyst.

24. The process of claim 22, wherein said first catalyst is different from said second catalyst.

25. The process of claim 22, said treatment is selected from the group consisting of the cracking of hydrocarbon feedstocks; the dewaxing of hydrocarbon feedstocks; the isomerization of xylenes; alkylation of aromatics; transalkylation of aromatics; toluene disproportionation; conversion of oxygenates to hydrocarbons; conversion of light paraffins and olefins to aromatics; and the desulfurization of an organic feed.

26. The process of claim 25, wherein the treatment conditions include a temperature from about 100° C. to about 760° C., a pressure of from about 0.1 atmosphere (bar) to about 200 atmospheres (bar), weight hourly space velocity of from about 0.08 hr⁻¹ to about 2000 hr⁻¹, and a hydrogen/organic molar ratio of from about 0 to about 100.

27. The process of claim 26, wherein the treatment is the desulfurization of a hydrocarbon-containing feed comprising benzene, toluene, or mixtures thereof.

28. The process of claim 27, wherein said first and second catalyst comprises an amorphous metal oxide and a hydrogenation/dehydrogenation metal.

29. The process of claim 16, wherein said distributor of said vertical reactor is capable of allowing removal of said first catalyst and said second catalyst from the bottom of said lower portion of said vertical reactor.
